(19) United States
(12) Patent Application Publication
(10) Pub. No.: US 2019/0117321 A1
(43) Pub. Date: Apr. 25, 2019

(54) ROBOTIC SYSTEM WITH END-EFFECTOR OVERHANG CONTROL

(71) Applicant: THINK SURGICAL, INC., Fremont, CA (US)

(72) Inventors: Youngbae Brian PARK, Fremont, CA (US); Joel ZUHARS, Fremont, CA (US)

(21) Appl. No.: 16/091,233

(22) PCT Filed: Mar. 17, 2017
(86) PCT No.: PCT/US2017/023060

§ 371 Date: Oct. 4, 2018

Related U.S. Application Data
(60) Provisional application No. 62/319,250, filed on Apr. 6, 2016.

(51) Int. Cl. A61B 34/30; A61B 17/14; A61B 17/16

(52) U.S. Cl. CPC A61B 34/30; A61B 2034/2055; A61B 17/16; A61B 17/14

(57) ABSTRACT

A system and process is provided for creating accurate and precise bone cuts with a tool, while maintaining the stability and mechanical integrity of the tool during bone cutting. In some instances, the tool is minimally invasive, where the use of a minimally invasive tool reduces the risk of injuring surrounding soft tissues and provides greater access to portions of the bony anatomy through a smaller surgical incision.

### CROSS-REFERENCE TO RELATED APPLICATION

This application claims priority of U.S. Provisional Patent Application Ser. No. 62/319,250 filed Apr. 6, 2016, which is incorporated herein by reference.

### TECHNICAL FIELD

The present invention generally relates to the field of robotic surgery, and more specifically to a system and process for adjusting or controlling the exposure and stiffness of an end-effector tool while executing a surgical procedure.

### BACKGROUND

Total knee arthroplasty (TKA) is a surgical procedure in which the articulating surfaces of the knee joint are replaced with prosthetic components. The distal femur and proximal tibia are prepared by cutting the bone to create planar surfaces, which receive the components in a final position and orientation (POSE). The final POSE of the components is important to restore the biomechanical alignment of the subject's leg, improve implant survivability, and reduce component wear. The precision and accuracy in the creation of the planar surfaces is critical, not only for the final component POSE, but also for the initial stability of cementless type components.

In order to create the aforementioned planar surfaces, various bone cutting strategies have been implemented. With reference to FIGS. 1A-1D, a bone B is shown having a targeted planar surface 102. In a conventional TKA procedure, with reference to prior art FIG. 1A, a surgeon may use a reciprocating saw 104 to create a planar surface 102, which is a fast but inaccurate method. To improve accuracy, with reference to prior art FIG. 1B, a cutting guide 106 with a guide slot 108 is used to guide and stabilize the blade of the reciprocating saw 104. The cutting guide 106 and guide slot 108 are aligned on the bone B using intramedullary and/or extramedullary alignment guides, which reference anatomical landmarks on the femur or tibia for alignment. However, the alignment of the cutting guide 106 can be inaccurate due to subject-to-subject variability, bone deformity, and the ability to correctly reference a given landmark.

Robotic surgical procedures have become a preferred medical technique for many complex surgeries which require a high level of precision. In general, the robotic surgical systems include two components, an interactive pre-operative planning software program and a robotic surgical device that utilizes the pre-operative data from the software to assist the surgeon in precisely executing the procedure.

Conventional interactive pre-operative planning software generates a three-dimensional (3-D) model of the subject's bony anatomy from a computed tomography (CT) or magnetic resonance imaging (MRI) image dataset of the subject. A set of 3-D computer aided design (CAD) models of the manufacturer's prosthesis are pre-loaded in the software that allows the user to place the components of a desired prosthesis to the 3-D model of the bony anatomy to designate the best fit, position, and orientation of the prosthesis to the bone. The user can then save the pre-operative planning data to an electronic medium that is loaded and read by the surgical device to assist the surgeon intra-operatively in executing the plan. Commercially available robotic systems for executing total and partial knee replacements include the TSolution One™ Surgical System (THINK Surgical, Inc., Fremont, Calif.) and the RIO® Interactive Orthopedic System (Stryker-Mako, Ft. Lauderdale, Fla.). Examples of these robotic systems are described in U.S. Pat. Nos. 5,086,401 and 7,206,626.

With reference to prior art FIGS. 1C and 1D, a robotic system may include an end-effector 110 that is removably attached to the distal end of a robotic manipulator arm. The end-effector may include a large diameter tool 112, as depicted in FIG. 1C, to create precise shapes in the bone. The large diameter tool 112 is stiff enough to cut the bone, but it may cause damage to the surrounding soft tissue at unexpected locations. The size of the large diameter tool 112 also requires a larger surgical incision to access the bones. With reference to prior art FIG. 1D, a relatively longer and smaller diameter tool 114 may provide greater access to portions of the knee through a smaller surgical incision, create more complex shapes, and reduce the risk of injuring the surrounding tissues. However, the smaller diameter tool 114 is prone to problems including: chattering, which results in an uneven surface finish; reduced stiffness, which can cause tool deflection; and tool breakage. Additionally, in general machining, to access deeper regions of the workpiece, tools of various lengths may be exchanged from the end-effector 110. But in an operating room, surgical time can be directly related to the amount of blood loss and potential infection, which discourages the swapping of tools from the end-effector 110 due to the time required for the swap.

Thus, there is a need for a system and process for creating precise and accurate cuts to a bone using a minimally invasive tool while maintaining the stability and mechanical integrity of the tool.

### SUMMARY OF THE INVENTION

An end-effector assembly is provided for a robotic surgical system to perform a procedure on a bone of a subject. The end-effector includes a tool with an elongated shaft, the elongated shaft having a working end and a proximal end; a motor configured to secure a portion of the proximal end of the elongated shaft; a sleeve axially supporting a portion of the elongated shaft; and an actuator in communication with at least one of the motor or the sleeve or a displaceable fluid source. The actuator imparts motion to the at least one of the motor or the sleeve in an axial direction of the tool to adjust the portion of the elongated shaft supported by the sleeve.

An end-effector assembly is provided for a robotic surgical system to perform a procedure on a bone of a subject. The end-effector includes: a tool with an elongated shaft, the elongated shaft having a working end and a proximal end; a motor configured to secure a portion of the proximal end of the elongated shaft; a sleeve axially supporting a portion of the elongated shaft; and an actuator in communication with at least one of the motor or the sleeve or a displaceable fluid source. The actuator imparts motion to the at least one of the motor or the sleeve in an axial direction of the tool for controlling stiffness of the tool.

A method is provided for using the end-effector as described, the method includes: creating a cut in the bone; and advancing the sleeve in the axial direction of the tool to control the stiffness of the tool based on a depth of cut into the bone.

A system is provided for using the end-effector described above, the system includes: a robot; a tracking system; a tool with an elongated shaft, the elongated shaft having a working end and a proximal end; a motor configured to secure a portion of the proximal end of the elongated shaft; a sleeve axially supporting a portion of the elongated shaft; and an actuator in communication with at least one of the motor or the sleeve displaceable fluid source. The actuator imparts motion to the at least one of the motor or the sleeve in an axial direction of the tool to adjust the portion of the elongated shaft supported by the sleeve. A housing is provided that encloses the motor, where the housing further includes a coupler for coupling the end-effector to a distal end of a robotic manipulator arm of the robot.

### BRIEF DESCRIPTION OF THE DRAWINGS

The present invention is further detailed with respect to the following drawings that are intended to show certain aspects of the present of invention, but should not be construed as a limit on the practice of the invention, wherein:

FIGS. 1A-1D depict prior art systems and processes for creating bone cuts, where FIG. 1A depicts the use of a reciprocating saw, FIG. 1B depicts the use of a reciprocating saw with a cutting guide, FIG. 1C depicts a robotic system end effector having a large diameter tool, and FIG. 1D depicts a robotic system end effector having a small diameter tool;

FIG. 2A illustrates an end-effector assembly having an actuator to adjust a tool between a retracted state as shown, in accordance with embodiments of the invention in the context of a native distal femur;

FIG. 2B illustrates the end-effector assembly of FIG. 2A in an extended state, and shown having modified the native femur;

FIG. 2C depicts a tool for use with the end-effector assembly;

FIGS. 2D and 2E depict a cross-sectional view of the end-effector assembly shown in FIGS. 2A and 2B having the actuator for adjusting the tool between a retracted state and extended state, respectively;

FIGS. 2F and 2G depict the end-effector assembly having the actuator where FIG. 2F corresponds to FIG. 2E and designates the location of a detailed view of a distal portion of the end-effector assembly as shown in FIG. 2G;

FIGS. 3A and 3B depict an end-effector assembly having a removable sleeve, with the sleeve on and off, respectively, in accordance with other embodiments of the invention;

FIGS. 3C and 3D depict a cross-sectional view of the end-effector assembly of FIG. 3A having a removable sleeve, with the sleeve on and off, respectively;

FIGS. 4A and 4B illustrate an end-effector assembly having an actuating sleeve in an extended and retracted position, respectively, in accordance with still other embodiments of the invention;

FIGS. 4C and 4D depict the end-effector assembly having an actuating sleeve, where FIG. 4C designates the location of a detailed view of a distal portion of the end-effector assembly shown in FIG. 4D in accordance with embodiments of the invention; and

FIG. 5 illustrates a robotic surgical system for use with the end-effector assembly in accordance with embodiments of the invention.

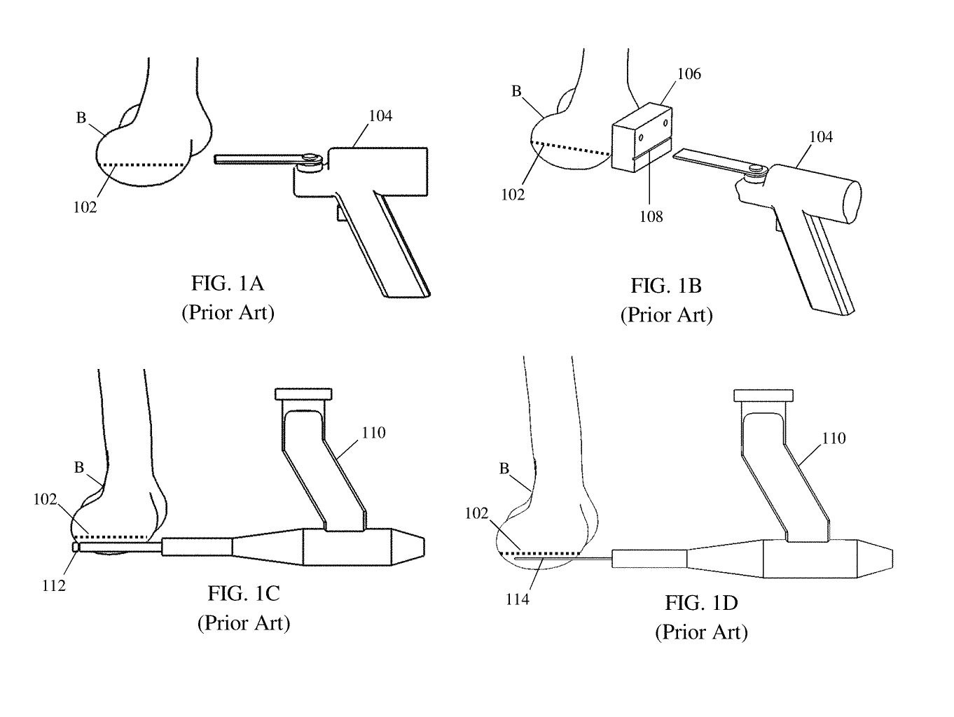

### DETAILED DESCRIPTION

The present invention has utility as a system and process for creating accurate and precise bone cuts with a tool, while maintaining the stability and mechanical integrity of the tool during bone cutting. In some instances, the tool is minimally invasive, where the use of a minimally invasive tool reduces the risk of injuring surrounding soft tissues and provides greater access to portions of the bony anatomy through a smaller surgical incision. The following description of the preferred embodiments of the invention is not intended to limit the invention to these preferred embodiments, but rather to enable any person skilled in the art to make and use this invention.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

As reference is made herein to total knee arthroplasty (TKA) as an applicable surgical procedure, it should be understood that the present invention is readily applied to other joints and bones found within the body. These other joints illustratively include the hip joint, shoulder joint, ankle joint, wrist joint, finger joint, toe joint, or other joints. In particular, the present invention is notably applicable for: creating planar cuts of the bone for osteotomy procedures; creating deep guide-holes; and creating narrow and deep slots for joint arthroplasty procedures such as TKA. As used herein, a subject is defined as a human, a non-human primate; or an animal of a horse, a cow, a sheep, a goat, a dog, a cat, a rodent and a bird; or a cadaver of any of the afore-mentioned.

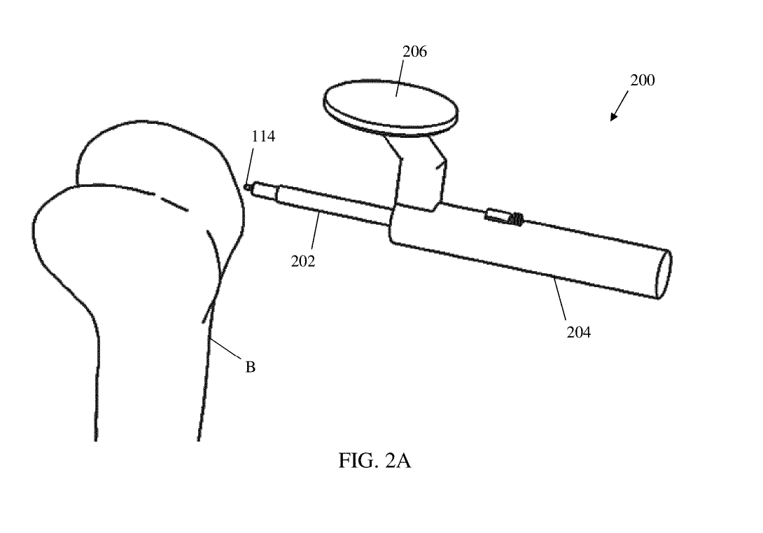

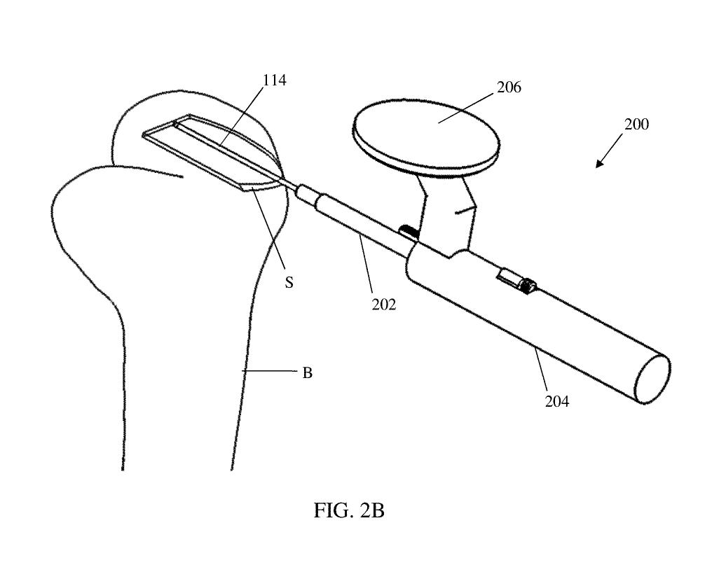

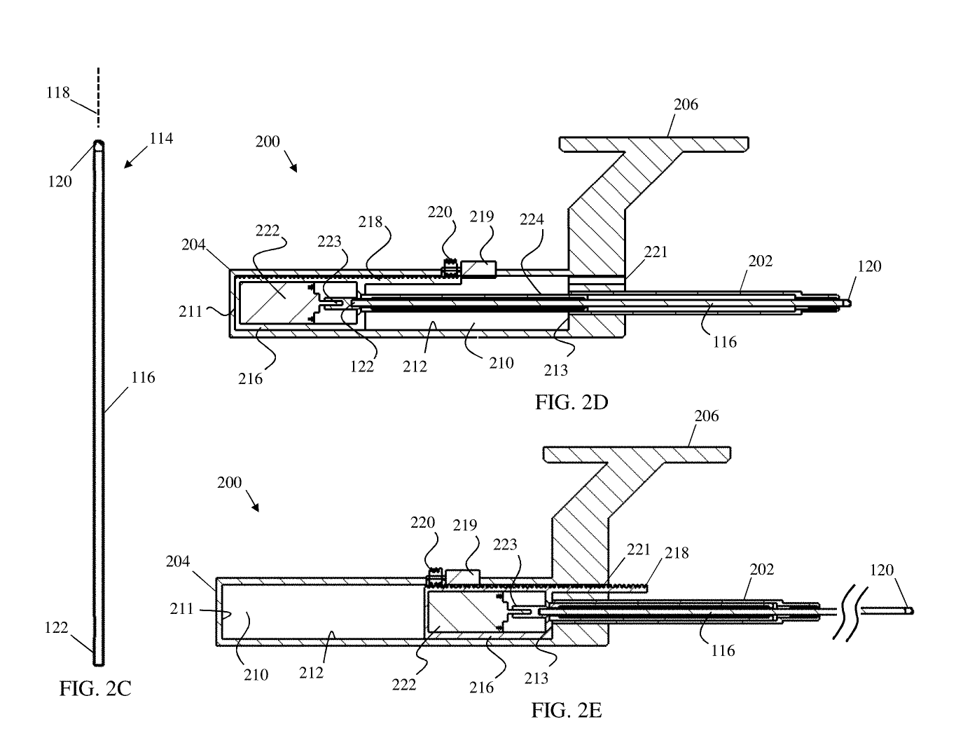

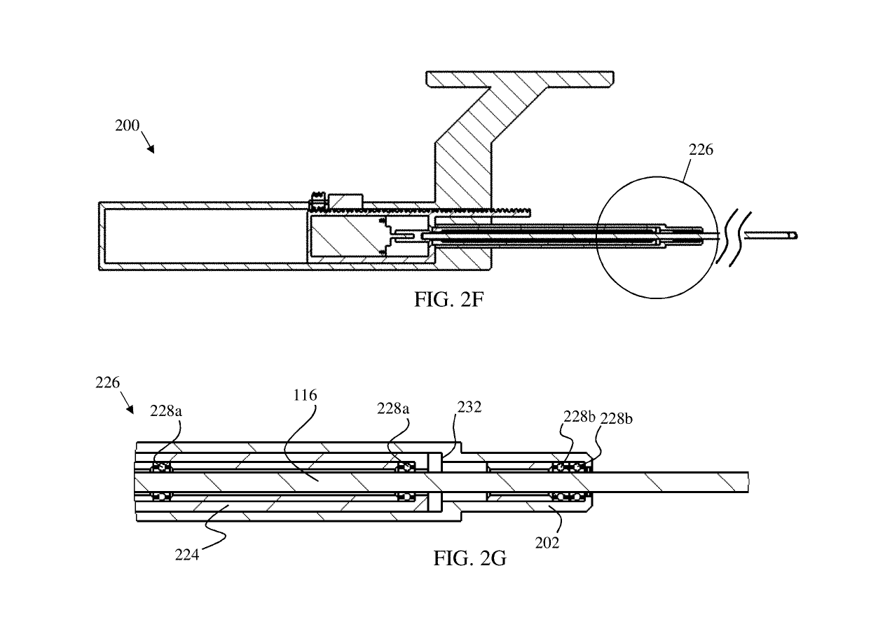

Embodiments of the present invention provide a robotic system and end-effector assembly for controlling or adjusting the exposure and stiffness of a minimally invasive tool for creating bone cuts. With reference to FIGS. 2A-2G, an inventive embodiment of an end effector assembly 200 is shown. As shown in FIG. 2A and 2B, the end-effector assembly 200 generally includes a minimally invasive tool 114, an outer sleeve 202 axially supporting the tool 114, an end-effector housing 204, and a mounting member 206. The end-effector assembly 200 can adjust the exposure and stiffness of the tool 114 between a fully retracted state, as shown in FIG. 2A, and a fully extended state, as shown in FIG. 2B. It is appreciated that intermediate states of extension also exist between those shown in FIGS. 2A and 2B. In the retracted state, the tool 114 is stiffer and less prone to chattering or tool deflection because the sleeve 202 is axially supporting a larger portion of the tool 114, as compared to the extended state. The retracted tool state may be best utilized for initially cutting the hard cortical bone. FIG. 2B depicts the tool 114 in the fully extended state cutting a slot S in the bone B. In the fully extended state, the tool 114 can access deeper regions in the bone B, but is less stiff as a greater portion of the tool 114 extends beyond the supporting sleeve 202. However, the inner trabecular bone is softer where tool deflection is of less concern, and where the chattering of the tool is suppressed by remnant bone above and below the slot S. The amount of tool exposure is readily adjusted between the fully retracted and extended state using embodiments of the end-effector assembly as further described below.

As shown in FIG. 2C, the tool 114 includes an elongated shaft 116 having an axial axis 118, a working end 120, and a proximal end 122. The working end 120 is designed to remove or cut bone, such as an end-mill, burr or other type of cutter design. The elongated shaft 116 may be provided in a variety of shapes including cylindrical, rectangular cuboid, or other polygonal cross sectional shape; and is made of stainless steel or an equivalent material conventional to the surgical tool manufacture. The proximal end 122 may also be cylindrical in shape or it is readily designed to be received in a special type of collet or chuck (e.g., a hex key and socket). In a particular embodiment, the elongated shaft 116, working end 120, and proximal end 122 are a monolithic structure made of the same materials. In another embodiment, the working end 120 may be welded to the elongated shaft 116. In a specific embodiment, the diameter of the working end 120 is no larger than the diameter of the elongated shaft 116. In another embodiment, the diameter of the working end 120 is greater than the diameter of the elongated shaft 116. In particular embodiments, the diameter of elongated shaft 116 and working end 120 are both independently between 1-5 millimeters.

FIGS. 2D and 2E provide cross-sectional views of the end-effector assembly 200 in a retracted state and an extended state, respectively. A portion of the end-effector housing 204 includes a chamber 210 enclosed by a proximal chamber surface 211, one or more lateral chamber surfaces 212, and a distal chamber surface 213. In a particular embodiment, the chamber is cylindrical having one continuous lateral chamber surface 212, although it should be appreciated that the chamber shape may vary, for example, the chamber 210 may be rectangular having four lateral chamber surfaces 212. A motor casing 216 may reside within a portion of the chamber 210, where the motor casing 216 can be reversibly actuated along the length of the one or more lateral chamber surfaces 212. A linear gear 218, such as a toothed rack gear, is attached or incorporated with at least a portion of the motor casing 216, where the teeth of the linear gear 218 project towards one or more of the lateral chamber surfaces 212. In a specific embodiment, the linear gear 218 spans at least a proximal-distal length of the motor casing 216. In another embodiment, the linear gear 218 may overhang beyond the proximal-distal length of the motor casing 216 to increase the amount the motor casing 216 may be actuated. The end-effector housing 204 may include a channel 221 to accommodate overhang of the linear gear 118 as best seen in FIG. 2E. An actuator 219 actuates the linear gear 218 and the motor casing 216. The actuator 219 having an actuator gear 220, such as a worm gear, is positioned on or with the end-effector housing 204 so that the actuator gear 220 may interlock and engage with the linear gear 218. For example, as shown in FIGS. 2D and 2E, the actuator gear 220 traverses through an outer surface of the end-effector housing 204 to the lateral chamber surface 212 to engage the linear gear 218. The actuator 219 can therefore operate the actuator gear 220 to reversibly actuate the linear gear 218 and motor casing 216 along the length of the one or more lateral chamber surfaces 212.

A motor 222 that drives the tool 114 is immovably encased within the motor casing 216 such that the motor 222 and tool 114 actuate in concert with the motor casing 216. A securing member 223, such as a collet or other chuck, may be used for removably securing the proximal end 122 of the tool 114 to the motor 222. The motor 222 can drive the tool 114 independent of the actuating motion of the motor casing 216, motor 222, and tool 114. In a particular embodiment, the end-effector assembly 200 may not include the motor casing 216, where the linear gear 218 is directly attached or integrated with the motor 222. It is appreciated that a pneumatic or hydraulic fluid being displaced in the chamber 210 supplants or acts as an adjunct to the actuator 219 to change the dynamic extension of the tool 114.

An outer sleeve 202 attached to or integrated with the end-effector housing 204 projects distally therefrom and provides an opening for the tool 114 to access the motor 222 within the chamber 210. In a specific inventive embodiment, the outer sleeve 202 is rigidly fixed to the end-effector housing 204 and axially supports a portion of the elongated shaft 116 of the tool 114. When the motor casing 216, motor 222 and tool 114 are actuated via the actuator 219, the portion of the elongated shaft 116 supported by the outer sleeve 202 changes. For example, when the tool 114 is in a retracted state the outer sleeve 202 supports the distal working end 120 of the elongated shaft 116, whereas when the tool 114 is in an extended state, the outer sleeve 202 only supports a more proximal portion of the elongated shaft 116.

In a particular embodiment, the end-effector assembly 200 includes an inner sleeve 224 projecting distally from the motor casing 216 to axially support a proximal portion of the elongated shaft 116. The inner sleeve 224 may be removably attached to or integrated with the motor casing 216 and actuates in concert with the motor casing 216 and the tool 114. The portion of the elongated shaft 116 supported by the inner sleeve 224 may be constant. It should be appreciated that the use of the inner sleeve 224 is not necessary as the outer sleeve 202 may have the ability to provide sufficient support for the tool 114, although additional support to the proximal portion of the elongated shaft 116 is provided with the use of the inner sleeve 224.

With respect to FIGS. 2F and 2G, FIG. 2F depicts the end-effector assembly 200 with the tool 114 in an extended state, and FIG. 2G provides a detailed view of the circled region 226 in FIG. 2F. In a specific embodiment, the inner sleeve 224 includes bearings 228a spaced along the axial length of the inner sleeve 224. Likewise, the distal end of the outer sleeve 202 may also include bearings 228b. The bearings 228a and 228b, such as ball bearings, is readily integrated with the inner sleeve 224 or outer sleeve 202 to fit about and directly interact with the elongated shaft 116 of the tool 114 so the tool 114 can freely rotate about the tool's axial axis 118 when driven by the motor 222. It is noted, that the tool 114 is readily removed or exchanged from the end-effector assembly 200 as needed or desired by un-securing the motor securing member 212 from the proximal end 122 of the tool 114, and sliding the tool 114 from of the inner sleeve 224 and/or outer sleeve 202.

In a particular embodiment, the inner diameter of the outer sleeve 202 may be greater than the outer diameter of the inner sleeve 224 such that the inner sleeve 224 may actuate within the outer sleeve 202. As illustrated in FIG. 2G, a distal segment of the outer sleeve 202 may have an inner diameter that is less than the outer diameter of the inner sleeve 224 to form an abutment surface 232 that restricts the inner sleeve 224 from actuating beyond the abutment surface 232. In this embodiment, only the distal end of the outer sleeve may include bearings 228b. In another embodiment, the end-effector assembly 200 lacks an inner sleeve 224, where the outer sleeve 202 may have a constant inner diameter with a plurality of bearings 228 spaced along the axial length of the outer sleeve 202.

### Removable Sleeve

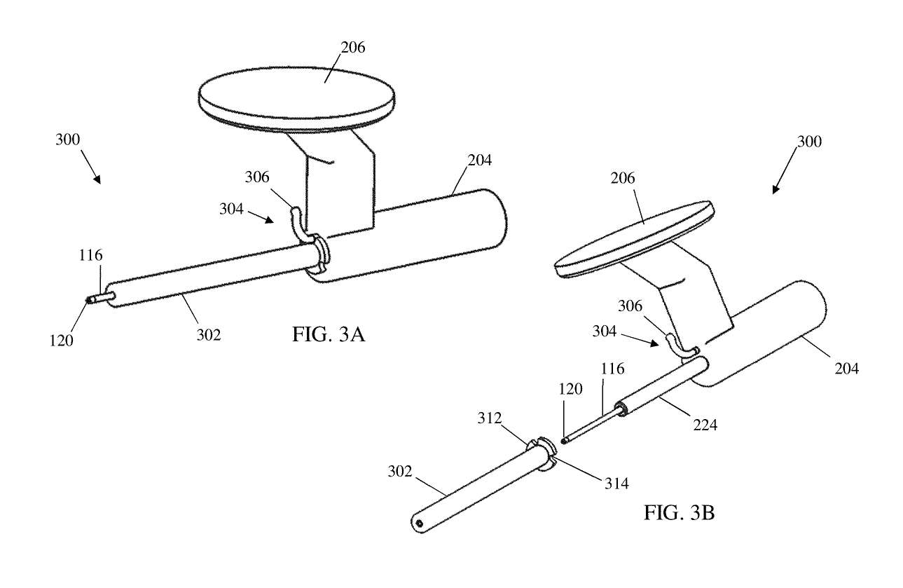

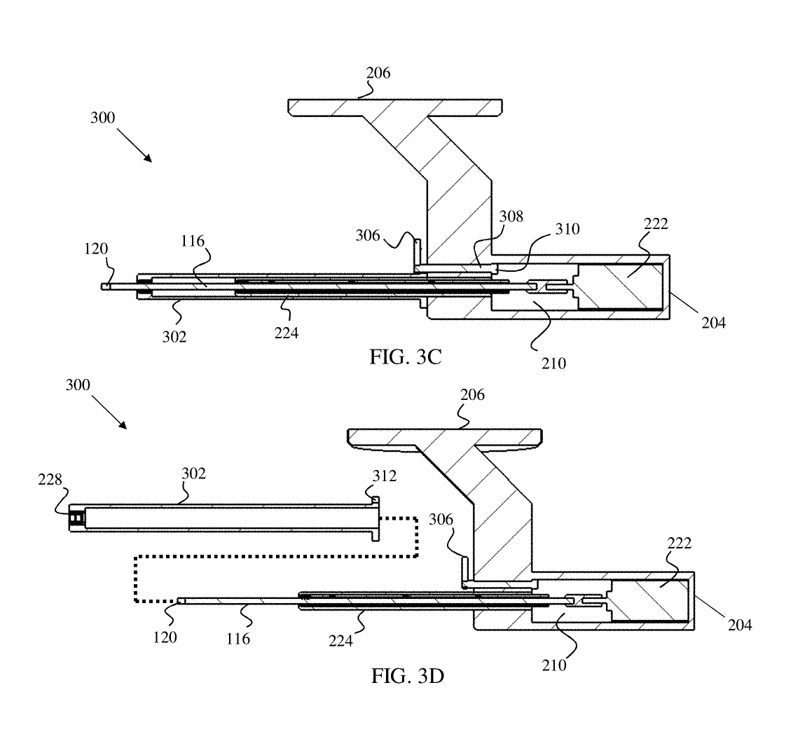

With reference to FIGS. 3A-3D in which like reference numerals have the meaning ascribed to that numeral with respect to the aforementioned figures, a particular embodiment of the end-effector assembly 300 includes a removable sleeve 302. In order to adjust the stiffness of the tool 114, the removable sleeve 302 is readily removably attached to the end-effector housing 204 to axially support a portion of the elongated shaft 116. FIG. 3A depicts the removable sleeve 302 attached to the end-effector housing 204 using a latch 304. The removable sleeve 204 may be used during the initial cutting of the hard cortical bone to reduce chatter and improve accuracy. With respect to FIG. 3B, once the initial cutting is complete, the removable sleeve 302 may be removed from the end-effector housing 204 so the tool 114 can access and cut deeper regions within the bone, such as trabecular bone, where chatter is suppressed by the remnant bone surrounding the tool 114. Additionally, the removable sleeve 302, which may contact the patient or bodily fluids, is readily cost-efficiently manufactured to be disposed after each use.

FIGS. 3C and 3D depict a cross-sectional view of the end-effector assembly 300 with the removable sleeve 302 attached and detached from the end-effector housing 204, respectively. The end-effector assembly 300 may or may not include an inner sleeve 224 for supporting a proximal portion 122 of the tool 114. One or more bearings 228 may be located at a distal end or spaced along the length of the removable sleeve 302. A proximal end of the removable sleeve 302 may include a rim 312 having one or more latch attachment points 314, such as indentations, depression or slots, to engage with the latch 304 and lock the removable sleeve 302 to the end-effector assembly 300. The latch 304 may include a latch rod 308 having a latching member 306 at a first end of the latch rod 308 and a latch retaining member 310 at a second end of the latch rod 308. The latch rod 308 may traverse through a distal portion of the end-effector housing 204 where the retaining member 310, such as a lip or edge, catches an inner region of the chamber 210 to prevent translation of the latch 304 relative to the end-effector housing 204. The latch 304 may be rotatable about the axial axis of the latch rod 308 to provide a mechanism for manually or automatically engaging or disengaging the latching member 306 to one or more of the latch attachment points 314 of the removable sleeve 302. Although, the act of attaching and detaching the removable sleeve 302 to the end-effector housing 204 is described with the use of the latch 304, other mechanisms, illustratively including magnets, clasps, couplers, threaded fastener, male-female connector, a press-fit, and equivalents thereof may accomplish the same.

In a specific embodiment, the end-effector assembly 300 with the removable sleeve 302 does not include the actuator 219 to actuate the tool 114 with respect to the end-effector housing 204, where the presence or absence of the removable sleeve 302 solely dictates the stiffness and exposure of the tool 114. Removable sleeves of varying length may also be used to vary the stiffness and exposure of the tool 114. In another embodiment, the end-effector assembly 300 with the removable sleeve 302 includes the actuation mechanisms as described in FIGS. 2A-2E to provide further adjustment of the exposure of the tool 114 from the removable sleeve 302.

### Actuating Sleeve

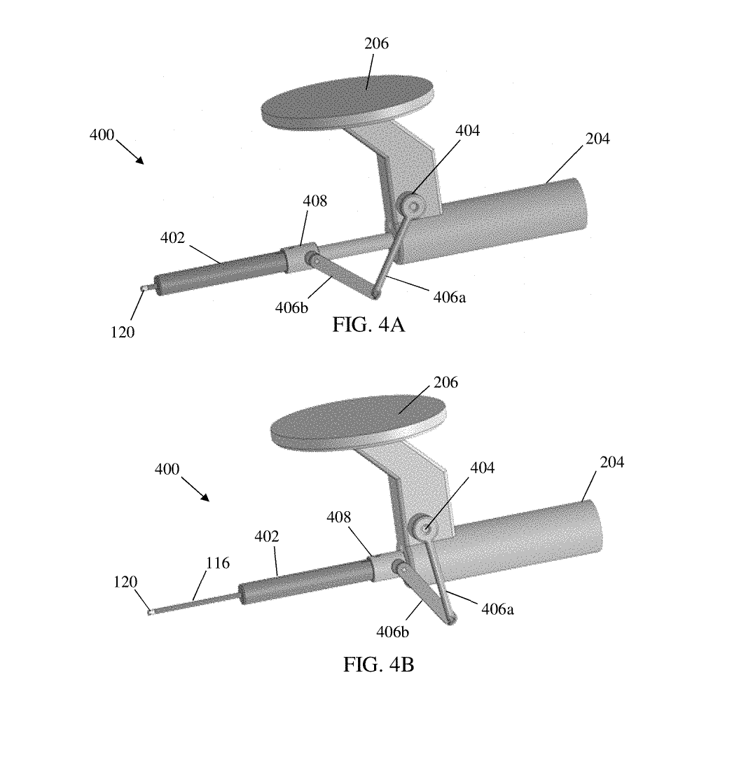

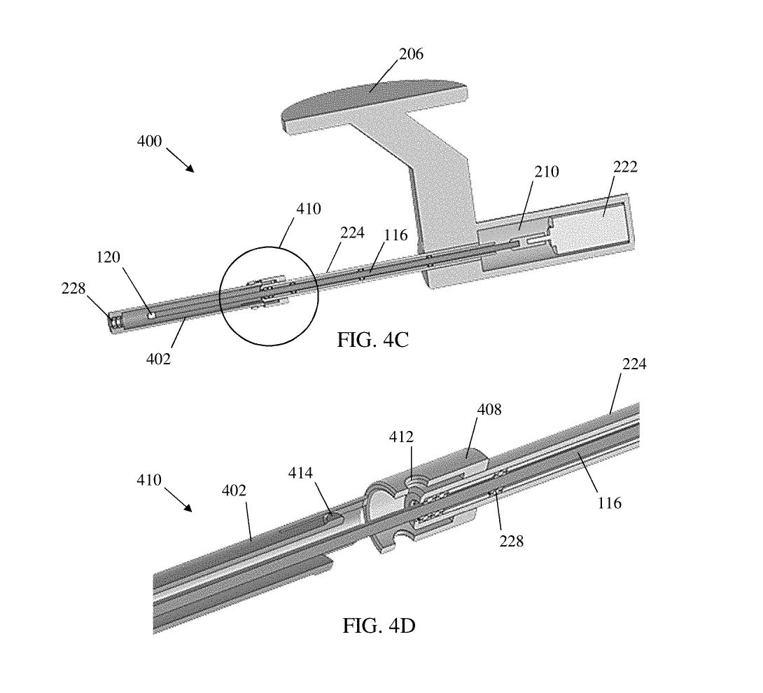

With reference to FIGS. 4A-4D in which like reference numerals have the meaning ascribed to the numeral with respect to the aforementioned figures, a particular inventive embodiment of the end-effector assembly 400 includes an actuating sleeve 402. FIG. 4A depicts the actuating sleeve 402 in an extended state and FIG. 4B depicts the actuating sleeve 402 in a retracted state. In a particular embodiment, a sleeve actuator 404 and a pair of linkages 406a and 406b are used to position of the actuating sleeve 402 to adjust the exposure and stiffness of the tool 114. The sleeve actuator 404 may be incorporated with the end-effector housing 204, where the actuator is in communication with a first end of a first link 406a. A proximal end of the actuating sleeve 402 includes a sleeve attachment member for operatively coupling a first end of a second link 406b. The opposing ends of the first link 406a and second link 406b are coupled by a joint. The actuator 404 and links (406a, 406b) are configured such that the actuation of the first link 406a causes the second link 406b to translate the sleeve 404 along the axial direction of the tool 114. It is appreciated that a pneumatic or hydraulic fluid being displaced in a chamber supplants or acts as an adjunct to the actuator 404 to change the dynamic extension of the actuating sleeve 402.

FIG. 4C is a cross-sectional view of the end-effector assembly 400, and FIG. 4D is a detailed view of the circled region 410 of FIG. 4C. In a specific embodiment, at least a portion of a sleeve attachment member 408 may surround and contact a portion of the inner sleeve 224 to provide support for a proximal portion of the actuating sleeve 402. A distal end of the actuating sleeve 402 may include bearings 228 as previously described. In a particular embodiment, the actuating sleeve 402 may be removable or replaceable from the sleeve attachment member 408. For example, the actuating sleeve 402 is readily translated to the distal end of the inner sleeve 228 as shown in detail in FIG. 4D, and removed from the attachment member 408. The attachment member 408 and the remaining actuating sleeve 402 may be removably attached with a quick connect mechanism, such as a bayonet connector, or other male-female connector. For instance, the attachment member 408 may include a circular opening 412 to receive a circular male counterpart 414 on the actuating sleeve 402.

In a specific embodiment, the end-effector assembly 400 with the actuating sleeve 402 does not include the actuator 219 to actuate the tool 114 with respect to the end-effector housing 204, where the translation of the actuating sleeve 402 along the length of the tool 114 solely dictates the stiffness and exposure of the tool 114. In another embodiment, the end-effector assembly 300 with the actuating sleeve 302 includes the actuation mechanisms as described in FIGS. 2A-2E to provide dual adjustment of the exposure and stiffness of the tool 114 from the actuating sleeve 302.

### Robotic Surgical System

Examples of robotic surgical systems that may utilize the end-effector assembly (200, 300, or 400) include a 1-6 degree of freedom hand-held surgical system, an autonomous serial-chain manipulator system, a haptic serial-chain manipulator system, a parallel robotic system, or a master-slave robotic system, as described in U.S. Pat. Nos. 5,086,401, 7,206,626, 8,876,830 and 8,961,536, U.S. Pat. App. No. 2013/0060278, and PCT Publication WO/2016/049180.

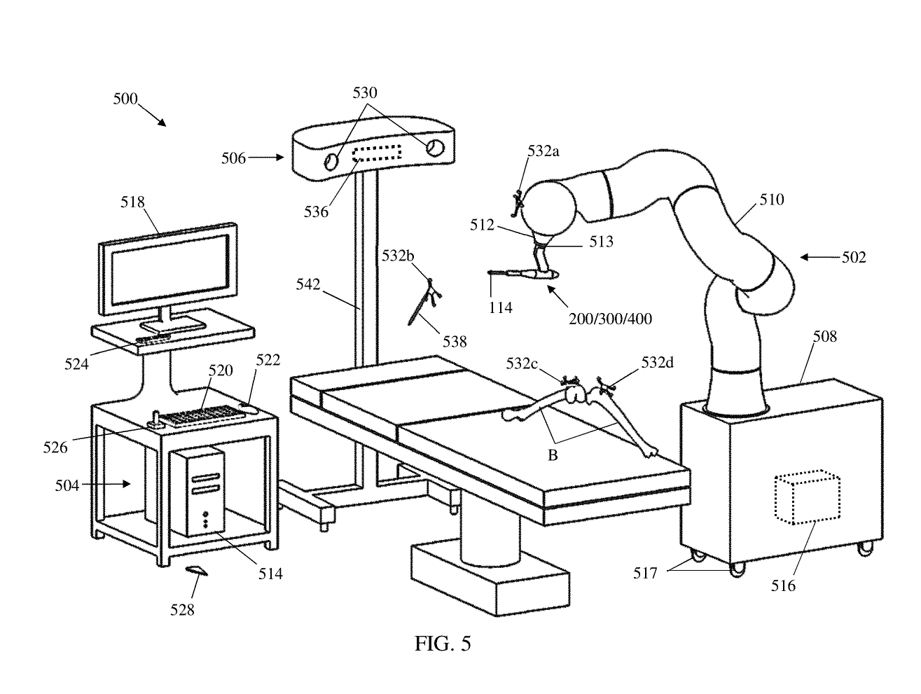

With reference to FIG. 5, a specific embodiment of a robotic surgical system 500 for a surgical procedure is shown in the context of an operating room (OR). The surgical system 500 generally includes a surgical robot 502, a computing system 504, and a tracking system 506.

The surgical robot 502 includes a movable base 508, a manipulator arm 510 connected to the base 508, an end-effector flange 512 located at a distal end of the manipulator arm 510, and the end-effector assembly 200, 300, 400 removably attached to the flange 512 by way of the end-effector mount 206. A force and torque sensor 513 is readily incorporated with the end-effector flange 512 or between the flange 512 and the end-effector assembly 200, 300, or 400 to measure forces and torques experienced by the tool 114 during the procedure. The base 508 may include a set of wheels 517 to maneuver the base 508, which may be fixed into position using a braking mechanism such as a hydraulic brake. The base may further include a prismatic joint to adjust the height of the manipulator arm 510. The manipulator arm 510 includes various joints and links to manipulate the tool 114 in various degrees of freedom. The joints may be prismatic, revolute, or a combination thereof. In a particular embodiment, the manipulator arm 510 and the tool's exposure and stiffness is controlled by commands from the computing system 502.

The computing system 502 generally includes a planning computer 514; a device computer 516; a tracking computer 536; and peripheral devices. Processors operate in system 502 to perform computations associated with the inventive method. It is appreciated that processor functions are shared between computers, a remote server, a cloud computing facility, or combinations thereof. The planning computer 514, device computer 516, and tracking computer 536 may be separate entities as shown, or it is contemplated that their operations may be executed on just one or two computers depending on the configuration of the surgical system 500. For example, the tracking computer 536 may have the operational data to control the manipulator 510 and actuation of the tool 114 without the need for a separate device computer 516. Or, the device computer 516 may include operational data to plan the surgical procedure without the need for a separate planning computer 514. In any case, the peripheral devices allow a user to interface with the surgical system components and may include: one or more user-interfaces, such as a display or monitor 518; and user-input mechanisms, such as a keyboard 520, mouse 522, pendent 524, joystick 526, foot pedal 528, or the monitor 518 may have touchscreen capabilities.

The planning computer 514 contains hardware (e.g., processors, controllers, and memory), software, data and utilities that are preferably dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan includes operational data for modifying a volume of tissue that is defined relative to the anatomy, such as a set of points in a cut-file to autonomously modify the volume of bone, a set of virtual boundaries defined to haptically constrain a tool within the defined boundaries to modify the bone, a set of planes or drill holes to drill pins in the bone, or a graphically navigated set of instructions for modifying the tissue. The surgical plan data may also include instructions for the end-effector assembly 200, 300 or 400 to control the exposure and stiffness of the tool 114 as the tool 114 modifies particular parts of the bone B (e.g., cortical bone, trabecular bone, deep bony regions). The data generated from the planning computer 514 is readily transferred to the device computer 516 and/or tracking computer 536 through a wired or wirelessly connection in the operating room (OR); or transferred via a non-transient data storage medium (e.g., a compact disc (CD), a portable universal serial bus (USB) drive) if the planning computer 514 is located outside the OR.

The device computer 516 may be housed in the moveable base 508 and contain hardware, software, data and utilities that are preferably dedicated to the operation of the surgical device 502. This may include surgical device control, robotic manipulator control, the processing of kinematic and inverse kinematic data, the execution of registration algorithms, the execution of calibration routines, the execution of surgical plan data, coordinate transformation processing, providing workflow instructions to a user, and utilizing position and orientation (POSE) data from the tracking system 506.

The tracking system 506 of the surgical system 500 includes two or more optical receivers 530 to detect the position of fiducial markers (e.g., retroreflective spheres, active light emitting diodes (LEDs)) uniquely arranged on rigid bodies. The fiducial markers arranged on a rigid body are collectively referred to as a fiducial marker array 532, where each fiducial marker array 532 has a unique arrangement of fiducial markers, or a unique transmitting wavelength/frequency if the markers are active LEDs. An example of an optical tracking system is described in U.S. Pat. No. 6,061,644. The tracking system 506 may be built into a surgical light, located on a boom, a stand 542, or built into the walls or ceilings of the OR. The tracking system computer 536 may include tracking hardware, software, data and utilities to determine the POSE of objects (e.g., bones B, surgical device 504) in a local or global coordinate frame. The POSE of the objects is collectively referred to herein as POSE data, where this POSE data is readily communicated to the device computer 516 through a wired or wireless connection. Alternatively, the device computer 516 may determine the POSE data using the position of the fiducial markers detected from the optical receivers 530 directly.

The POSE data is determined using the position data detected from the optical receivers 530 and operations/processes such as image processing, image filtering, triangulation algorithms, geometric relationship processing, registration algorithms, calibration algorithms, and coordinate transformation processing. For example, the POSE of a digitizer probe 538 with an attached probe fiducial marker array 532b may be calibrated such that the probe tip is continuously known as described in U.S. Pat. No. 7,043,961. The POSE of the tool tip or tool axis of the tool 114 may be known with respect to a device fiducial marker array 532a using a calibration method as described in U.S. Prov. Pat. App. 62/128,857. The device fiducial marker 532a is depicted on the manipulator arm 510 but may also be positioned on the base 508 or the end-effector assembly 200, 300, or 400. Registration algorithms is readily executed to determine the POSE and/or coordinate transforms between a bone B, a fiducial marker array (532a, 532b, 532c, 532d), a surgical plan, and/or the robotic system 502 using the registration methods described in U.S. Pat. Nos. 6,033,415, and 8,287,522.

Upon assembly of the device tracking array 532a to the surgical robot 502 prior to surgery, the POSE's of the coordinate systems, 532a and 114, are fixed relative to each other and stored in memory to accurately track the end effector tool 114 during the surgery (see for example U.S. Patent Publication No. 2014/0039517 A1) relative to the bone anatomy (e.g., bones B). The POSE data may be used by the computing system 504 during the procedure to update the bone and surgical plan coordinate transforms so the surgical robot 502 can accurately execute the surgical plan in the event any bone motion occurs. It should be appreciated that in certain embodiments, other tracking systems may be incorporated with the surgical system 500 such as an electromagnetic field tracking system or a mechanical tracking system. An example of a mechanical tracking system is described in U.S. Pat. No. 6,322,567. In a particular embodiment, the surgical system 500 does not include a tracking system 506, but instead employs a bone fixation and monitoring system that fixes the bone directly to the surgical robot 502 and monitors bone movement as described in U.S. Pat. No. 5,086,401 incorporated by reference herein in its entirety.

As the robotic surgical system performs the surgical procedure on the bones B, the end-effector assembly 200, 300, or 400 the exposure and stiffness of the tool 114 can be adjusted accordingly using the mechanisms as described above. An electrical connection between the end-effector mount 206 and the flange 512 may be established to send/receive signals between the device computer 516 and the actuator 219 or sleeve actuator 404. The actuator 219 and/or sleeve actuator 404 may also receive signals from the computing system wirelessly. Additionally, the adjustments are readily solely controlled manually by a user using the removable sleeve 302. In a particular embodiment, the adjustments are controlled by commands generated from the computing system 504. In one example, the surgical plan may include instructions for the end-effector assembly 200, 300, 400 to adjust the stiffness based on the position of the tool 114 with respect to the bony anatomy. For example, when the tool 114 is cutting the initial cortical bone, the plan may indicate the tool 114 to be in a more retracted state. Once the initial cutting is complete and the tool 114 is positioned beyond the cortical bone, the plan may instruct the actuator 219 or the sleeve actuator 404 to increase the exposure of the tool 114. In another example, the surgical plan data may include relative density values of the bone B being cut. The density values may be extracted from the medical imaging data such computed tomography (CT), magnetic resonance imaging (MRI), or dual-energy X-Ray absorptiometry (DEXA) image data sets of the bone B. The stiffness and exposure length of the tool may then be controlled based on the relative density values where higher values require greater stiffness compared to less dense regions.

In a particular embodiment, the force and torque sensor 513 may monitor the forces experienced by the tool 114 during cutting. The amount of exposure and stiffness of the tool 114 are readily correlated to the recorded forces. Maximum, minimum, or ranges of forces are readily established to provide a reference for determining the amount of stiffness or exposure required while cutting. This may provide real-time control of the tool 114 without any additional data from the surgical plan. In another embodiment, a combination of surgical plan data and real-time force data may be used for controlling the stiffness or exposure of the tool 114 during the procedure.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. An end-effector assembly for a robotic surgical system to perform a procedure on a bone of a subject, said end-effector comprising:
a tool with an elongated shaft, said elongated shaft having a working end and a proximal end;
a motor configured to secure a portion of the proximal end of said elongated shaft;
a sleeve axially supporting a portion of said elongated shaft; and
an actuator in communication with at least one of said motor or said sleeve or a displaceable fluid source, said actuator imparting motion to the at least one of said motor or said sleeve or said displaceable fluid source in an axial direction of said tool to adjust the portion of said elongated shaft supported by said sleeve.

2. The end-effector assembly of claim 1 further comprising a housing and a coupler, said housing enclosing said motor and said coupler mechanically joining said end-effector to a distal end of a robotic manipulator arm.

3. The end-effector assembly of claim 1 wherein a working end diameter of said working end radial axis is no larger than an elongated shaft diameter of said elongated shaft.

4. An end-effector assembly for a robotic surgical system to perform a procedure on a bone of a subject, said end-effector comprising:
a tool comprising an elongated shaft, said elongated shaft having a working end and a proximal end;
a motor configured to secure a portion of the proximal end of said elongated shaft;
a sleeve axially supporting a portion of said elongated shaft; and
an actuator in communication with at least one of said motor or said sleeve or a displaceable fluid source, said actuator imparting motion to the at least one of said motor or said sleeve in an axial direction of said tool for controlling stiffness of said tool.

5. A method of using the end-effector of claim 1, said method comprising:
creating a cut in the bone; and
advancing said sleeve in the axial direction of said tool to control the stiffness of said tool based on a depth of cut into the bone.

6. The method of claim 5 further comprising discarding said sleeve after the procedure.

7. The method of claim 5 further comprising;
detecting a force, in real-time, from a force sensor incorporated with said end-effector or said manipulator arm; and
advancing said sleeve in the axial direction of said tool to control the stiffness of said tool based on a range of force values that represent a cancellous bone type.

8. A system for using the end-effector of claim 1, said system comprising:
a robot;
a tool with an elongated shaft, said elongated shaft having a working end and a proximal end;
a motor configured to secure a portion of the proximal end of said elongated shaft;
a sleeve axially supporting a portion of said elongated shaft; and
an actuator in communication with at least one of said motor or said sleeve displaceable fluid source, said actuator imparting motion to the at least one of said motor or said sleeve in an axial direction of said tool to adjust the portion of the elongated shaft supported by said sleeve; and
a housing enclosing said motor, wherein said housing further comprises a coupler for coupling said end-effector to a distal end of a robotic manipulator arm of said robot.

9. The system of claim 8 further comprising a tracking system for tracking said tool relative to said bone, said tracking system providing control commands to said actuator based on the position of said tool within said bone.

10. The system of claim 8 further comprising a fixator for coupling said bone in a known position with respect to said robot and wherein said robot provides control commands to said actuator based on the position of said tool within said bone.

11. The system of claim 8 further comprising a force sensor incorporated with said end-effector or said manipulator arm to detect, in real-time, forces experienced by the tool and wherein said robot provides control commands to said actuator based on the detected forces.
